(19) United States
(12) Patent Application Publication
(10) Pub. No.: US 2026/0275588 A1
(43) Pub. Date: Sep. 17, 2026

(54) FUNCTIONAL FABRIC

(71) Applicant: MAS Innovation (Private) Limited, Battaramulla (LK)

(72) Inventors: Mapitiyage Don Janith DUSHYANTHA, Battaramulla (LK); Jithmi Mayondi DAYARATNE, Battaramulla (LK); Hettiarachchige Aasie Ayanthika WIJESINGHE, Battaramulla (LK); Kirivitige Asel Sathsara FERNANDO, Battaramulla (LK)

(21) Appl. No.: 19/665,671

(22) Filed: May 1, 2026

Related U.S. Application Data
(63) Continuation of application No. PCT/SG2024/050511, filed on Aug. 12, 2024.

(51) Int. Cl. D03D 15/527; D03D 15/54

(52) U.S. Cl. CPC D03D 15/527; D03D 15/54; D10B 2401/021; D10B 2401/022; D10B 2501/00

(57) ABSTRACT

The present disclosure generally relates to a functional fabric for use in a garment. The functional fabric comprises first hydrophobic yarns and second hydrophilic yarns arranged to form a composite fabric layer. The composite fabric layer includes a user facing side and an outer facing side. The outer facing side of the composite fabric layer includes a substantially liquid-impermeable barrier.

### CROSS-REFERENCE TO RELATED APPLICATIONS

This Application is a Continuation of International Patent Application No. PCT/SG2024/050511, filed on Aug. 12, 2024. The entire content of the above International Patent Application is incorporated herein by reference.

### TECHNICAL FIELD

### Field

The present disclosure generally relates to a functional fabric, which may form a functional portion of a garment, and a garment comprising the functional fabric. More particularly, the present disclosure describes various embodiments of the functional fabric for use in a garment, such as an undergarment or panty, as well as the garment comprising the functional fabric. The functional fabric may comprise an absorbent fabric and/or a wicking fabric. The present disclosure also relates to stain-resistant, stain-release, and/or anti-stain fabrics.

### BACKGROUND

Absorbent fabrics may be incorporated into garments, such as undergarments. Absorbent garments such as reusable and washable absorbent undergarments are worn with the purpose of absorbing bodily fluids. These bodily fluids include vaginal discharge, urine, menstrual fluid, sweat, and breast milk. Many people suffer from involuntary excretion of bodily fluids and there are few garments that have been designed to absorb such excretions that are functional and comfortable to the user wearing it. Absorbent garments may also be prone to staining by bodily fluids.

For example, a woman who is menstruating will generally use a tampon or a sanitary pad, in addition to wearing an undergarment, to keep her outer garments from being soiled by menstrual fluid. While the tampon or pad often absorbs all the liquid flow, unexpected leaks can still occur. To avoid such leaks, she can instead choose to wear an adult brief, which offers a larger area of protection and may be particularly useful for women experiencing heavy menstrual flows. Adult briefs may also be useful for people who have urinary incontinence. However, adult briefs tend to be bulky and unattractive, making it difficult to conceal them under outer clothing, which may cause embarrassment to the user. Some disadvantages of wearing adult briefs and sanitary pads include prolonged exposure to wetness, which may result in discomfort, irritant dermatitis, and/or infections. Further, pads / tampons may be occasionally positioned incorrectly and adult briefs may be wrapped too loosely, both resulting in leakage. In addition, these conventional products are generally disposable, meaning that the environmental and economic costs can be significant.

In general, stain-release or anti-stain fabrics may comprise hydrophobic materials or surfaces, which may be completely hydrophobic, to avoid the forming of bonds and hence avoid staining. However, the completely hydrophobic nature of prior stain-resistant fabrics limits the ability for these materials to absorb or wick liquids or fluids.

Therefore, in order to address or alleviate at least one of the aforementioned problems and/or disadvantages, there is a need to provide an improved absorbent fabric, or absorbent portion of a garment and an improved garment comprising the absorbent fabric.

### SUMMARY

According to a first aspect of the present disclosure, there is disclosed a functional fabric for use in a garment. The functional fabric comprises:

first hydrophobic yarns and second hydrophilic yarns arranged to form a composite fabric layer, the composite fabric layer comprising a user facing side and an outer facing side, and

wherein the outer facing side comprises a substantially liquid-impermeable barrier.

In some embodiments, the functional fabric forms a part or a portion of a garment or an undergarment.

In some embodiments, the functional fabric forms at least part of a gusset of an undergarment.

In some embodiments, the functional fabric is substantially leak proof or liquid impermeable.

In some embodiments, the functional fabric further comprises a liquid impermeable barrier layer or hydrophobic print disposed on at least a portion of the outer facing side of the composite fabric layer to form the substantially liquid-impermeable barrier.

In some embodiments, the composite fabric layer is arranged such that the user facing side comprises an arrangement of first hydrophobic yarns and second hydrophilic yarns, and the outer facing side comprises substantially hydrophobic yarns or only hydrophobic yarns.

In some embodiments, the composite fabric layer comprises a ratio of hydrophilic yarns to hydrophobic yarns.

In some embodiments, the ratio of hydrophilic yarns to hydrophobic yarns forming the composite fabric layer is about 50/50, or wherein the quantity of hydrophilic yarn is between about 20% and about 80% of the composite fabric.

In some embodiments, the outer facing side of the composite fabric layer comprises a higher ratio of hydrophobic yarns to hydrophilic yarns than the user facing side.

In some embodiments, the ratio of hydrophilic yarns to hydrophobic yarns comprises a varying gradient extending from the first user facing side to the second outer facing side.

In some embodiments, the functional fabric further comprises a hydrophobic print provided on at least a portion of the user facing side.

In some embodiments, the composite fabric layer comprises a knit or weave of hydrophobic yarns and hydrophilic yarns.

In some embodiments, the composite fabric layer comprises a pattern of alternating hydrophobic and hydrophilic yarns.

In some embodiments, the composite fabric layer comprises a linear array of hydrophobic yarns and hydrophilic yarns.

In some embodiments, the composite fabric layer comprises alternating lines of hydrophobic yarns disposed on top of or within the hydrophilic yarns

In some embodiments, the composite fabric layer comprises an array of hydrophobic yarns and hydrophilic yarns that form channels for guiding liquid flow.

In some embodiments, the user facing side comprises an array of channels formed of alternating hydrophobic yarns and hydrophilic yarns for guiding liquid flow, the channels comprising a region of hydrophobic yarns confined by one or more regions of hydrophilic yarns.

In some embodiments, the user facing side is formed of the hydrophilic yarns, and comprises alternating lines of hydrophobic yarns disposed on top of or within the hydrophilic yarns.

In some embodiments, the composite fabric layer comprises islands formed of hydrophobic yarns within areas of hydrophilic yarns.

In some embodiments, the composite fabric layer provides both a wicking and an absorbent function.

In some embodiments, the functional fabric is configured such that a colour of the hydrophilic yarns varies compared to a colour of the hydrophobic yarns when the functional fabric absorbs a liquid.

In some embodiments, the hydrophilic yarns comprise a cotton yarn.

In some embodiments, the hydrophobic yarns comprise pre-treated cotton yarns (pre-treated before a forming step of the composite material).

In some embodiments, the pre-treated cotton yarns are pre-treated with a chemical treatment (optionally a hydrophobic coating).

In some embodiments, the hydrophilic yarns provide for a wicking and/or absorbing region of the composite fabric layer.

In some embodiments, the hydrophobic yarns (or a distribution of the hydrophobic yarns, or an arrangement of the hydrophobic yarns and the hydrophilic yarns) provide for a stain releasing function of the composite layer.

In some embodiments, the functional fabric is arranged to provide for a stain release or stain-resistant or anti-stain fabric.

In some embodiments, wherein the user facing side provides for a stain release function.

In some embodiments, the functional fabric comprises an absorbent fabric or absorbent function.

According to a second aspect of the present disclosure, there is disclosed a stain-resistant, stain-release, or anti-stain fabric, comprising:

a first hydrophobic portion, and

a second hydrophilic portion,

wherein the stain-resistant or anti-stain fabric comprises a user facing side, and an outer facing side, and

wherein an outer facing side of the fabric comprises a substantially liquid-impermeable barrier.

In some embodiments, the first and second portions are alternated within the fabric.

In some embodiments, the stain-resistant fabric forms a part or a portion of a garment or an undergarment, or wherein the stain-resistant fabric forms at least part of a gusset of an undergarment.

In some embodiments, the stain-resistant fabric is substantially leak proof or liquid impermeable.

In some embodiments, the first hydrophobic portion is positioned on a user facing side of the stain-resistant fabric as a liquid impermeable barrier layer or hydrophobic print disposed on at least a portion of the user facing side.

In some embodiments, the second hydrophilic portion comprises hydrophilic yarns forming a portion of the stain-resistant fabric.

In some embodiments, the first hydrophobic portion comprises hydrophobic yarns, and the second hydrophilic portion comprises hydrophilic yarns.

In some embodiments, the stain-resistant fabric comprises a ratio of hydrophilic yarns to hydrophobic yarns.

In some embodiments, the ratio of hydrophilic yarns to hydrophobic yarns forming the stain-resistant fabric is about 50/50, or wherein the quantity of hydrophilic yarn is between about 20% and about 80% of the stain-resistant fabric.

In some embodiments, the stain-resistant fabric is arranged such that the user facing side comprises an arrangement of hydrophobic yarns and hydrophilic yarns, and the outer facing side comprises substantially hydrophobic yarns or only hydrophobic yarns.

In some embodiments, the stain-resistant fabric comprises a composite fabric layer, and wherein the composite fabric layer comprises the first hydrophobic portion and the second hydrophilic portion.

In some embodiments, the stain-resistant fabric is configured such that a colour of the second hydrophilic portion varies compared to a colour of the first hydrophobic portion when the stain-resistant fabric absorbs a liquid.

In some embodiments, the user facing side comprises an array of channels formed of alternating regions of the first hydrophobic portion and the second hydrophilic portion

In some embodiments, the channels comprise respective regions of the first hydrophobic portion confined by one or more regions of the second hydrophilic portion.

According to a third aspect of the present disclosure, there is disclosed a functional fabric for use in a garment, the functional fabric comprising:

one or more hydrophobic regions, and one or more hydrophilic regions, the hydrophobic region(s) and hydrophilic region(s) arranged to form a composite fabric layer, the composite fabric layer comprising a user facing side and an outer facing side, and

a liquid impermeable barrier layer or hydrophobic print disposed on at least a portion of the outer facing side of the composite fabric layer,

wherein the liquid impermeable barrier layer of hydrophobic print forms a substantially liquid-impermeable barrier, to prevent or reduce seeping of a fluid through the outer facing side of the composite fabric layer.

In some embodiments, when the functional fabric is incorporated with, or to, a garment, the liquid impermeable barrier layer prevents or reduces seeping of a fluid into the garment.

Set out above are various aspects and embodiments of the invention as presently disclosed. It will be understood that any of the aspects or embodiments disclosed in relation to aspects one, two or three may be reasonably combined to fall within the scope of the invention.

A functional fabric for use in a garment, a garment comprising the functional fabric, and a stain-resistant, stain release, or anti-stain fabric according to the present disclosure are thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1A illustrates a functional fabric or portion of a garment, in accordance with embodiments of the present disclosure.

FIG. 1B illustrates a garment comprising the functional fabric in accordance with embodiments of the present disclosure.

FIG. 2 illustrates an example composite fabric layer forming part of a functional fabric or portion of a garment in accordance with embodiments of the present disclosure.

FIGS. 3A to 3D illustrate various example functional fabric arrangements in accordance with embodiments of the present disclosure.

FIGS. 4 illustrates an example functional fabric arrangement in accordance with embodiments of the present disclosure.

FIG. 5 illustrates an example functional fabric arrangement in accordance with embodiments of the present disclosure.

FIG. 6 illustrates an example functional fabric arrangement in accordance with embodiments of the present disclosure.

FIG. 7A illustrates an example functional fabric arrangement in accordance with embodiments of the present invention.

FIG. 7B illustrates experimental results of the example arrangement of FIG. 7A.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

For purposes of brevity and clarity, descriptions of embodiments of the present disclosure are directed to a functional fabric for use in a garment and a garment comprising the functional fabric, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognised by an individual having ordinary skill in the art, i.e. a skilled person, that the present disclosure may be practiced without specific details, and/or with multiple details arising from combinations of aspects of particular embodiments. In a number of instances, known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment / example”, “another embodiment / example”, “some embodiments / examples”, “some other embodiments / examples”, and so on, indicate that the embodiment(s) / example(s) so described may include a particular feature, structure, characteristic, property, element, or limitation, but that not every embodiment / example necessarily includes that particular feature, structure, characteristic, property, element, or limitation. Furthermore, repeated use of the phrase “in an embodiment / example” or “in another embodiment / example” does not necessarily refer to the same embodiment / example.

The terms “comprising”, “including”, “having”, and the like do not exclude the presence of other features / elements / steps than those listed in an embodiment. Recitation of certain features / elements / steps in mutually different embodiments does not indicate that a combination of these features / elements / steps cannot be used in an embodiment. The terms “a” and “an” are defined as one or more than one. The use of “/” in a figure or associated text is understood to mean “and/or” unless otherwise indicated. The recitation of a particular numerical value or value range herein is understood to include or be a recitation of an approximate numerical value or value range.

The term “set” is defined as a non-empty finite organisation of elements that mathematically exhibits a cardinality of at least one (e.g. a set as defined herein can correspond to a unit, singlet, or single-element set, or a multiple-element set), in accordance with known mathematical definitions. The terms “first”, “second”, etc. are used merely as labels or identifiers and are not intended to impose numerical requirements on their associated terms.

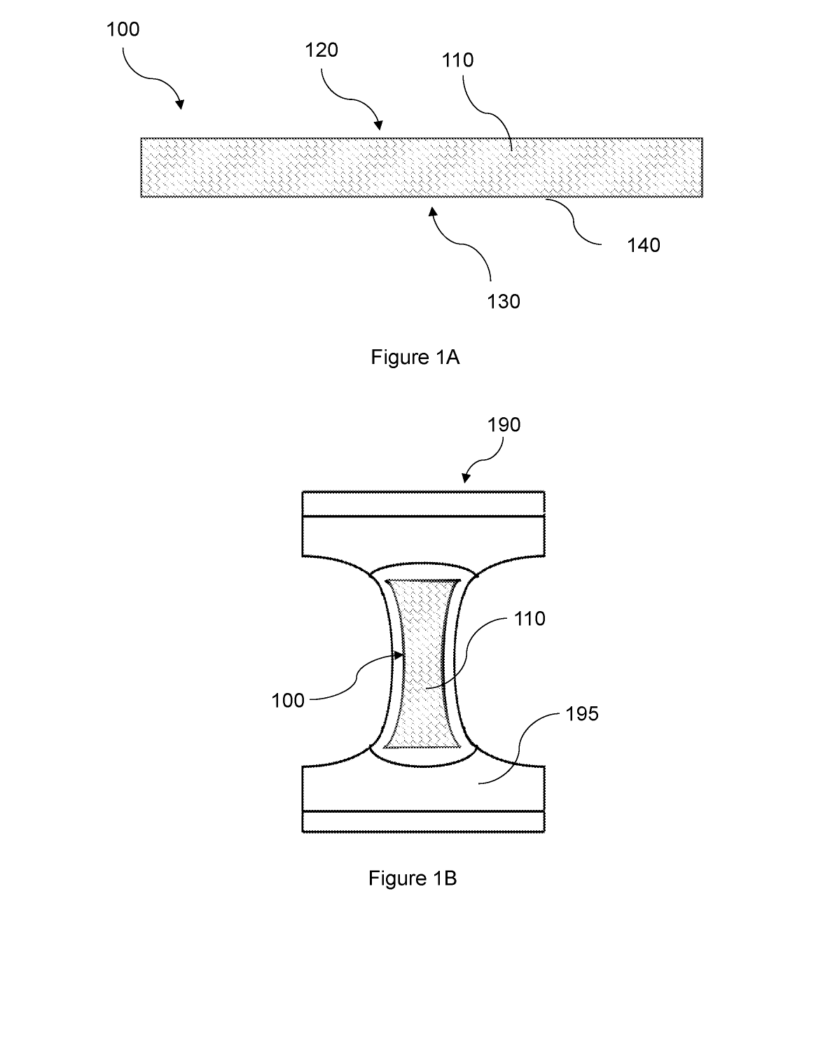

In representative or exemplary embodiments of the present disclosure, there is a functional fabric 100, which may form a portion of a garment 190, as illustrated in FIGS. 1A and 1B. The functional fabric 100 may comprise an absorbent fabric 100. FIG. 1A illustrates a cross section of the functional fabric 100, and FIG. 1B illustrates a top down view of the functional fabric 100 forming a portion of a fabric body 195 of the garment 190. The garment 190 may be an intimate garment or undergarment worn by a user particularly at parts of the body where there may be excretions of bodily fluids. For example, the garment 190 may be, but is not limited to, brassieres, panties, lingerie, active wear, athleisure wear, sportswear, swimwear, and similar close-fitting or form-fitting garments.

The functional fabric 100 may comprise a fabric layer 110, which may comprise a composite fabric layer. The functional fabric 100 and/or the composite fabric layer 110 may comprise a user facing side or surface 120, and an outer facing side or surface 130. In an example embodiment, the functional fabric 100 may form part of, or be attached to, a garment 190 to be worn by a subject or user, where the user facing side 120 is a side facing a user or subject when the garment 190 comprising the functional fabric 100 is in use. Similarly, the outer facing side 130 may be a side of the garment 190 comprising the functional fabric 100 opposite to the user facing side 120, and facing outwards away from a user when a garment 190 comprising the functional fabric 100 is in use.

The functional fabric 100 may comprise a substantially liquid impermeable barrier 140. The liquid impermeable barrier 140 may form the outer facing side 130 of the functional fabric 100, or may be positioned in a region of or attached to the outer facing side 130. The liquid impermeable barrier 140 may be substantially, partially, or completely impermeable to liquids passing through the barrier.

In an example embodiment as shown in FIG. 1B, a functional fabric 100 may be incorporated with, or to a garment 190. As disclosed herein, the functional fabric 100 may comprise one or more hydrophobic regions, and one or more hydrophilic regions, the hydrophobic region(s) and hydrophilic region(s) arranged to form a composite fabric layer 110, the composite fabric layer comprising a user facing side 120 and an outer facing side 130. The functional fabric may comprise a liquid impermeable barrier layer or hydrophobic print, represented by numeral 140, that may be disposed on at least a portion of the outer facing side 130 of the composite fabric layer 110. In an example embodiment, the functional fabric 100 may be configured such that the liquid impermeable barrier layer or hydrophobic print forms a substantially liquid-impermeable barrier, to prevent or reduce seeping of a fluid through the outer facing side 130 of the composite fabric layer 110. In an example embodiment, for example when the functional fabric 100 is incorporated with, or to, a garment 190, this may advantageously provide for a liquid impermeable barrier layer formed on or as part of the composite layer 110 and/or the functional fabric 100 that may prevent or reduce seeping of a fluid into the garment 190.

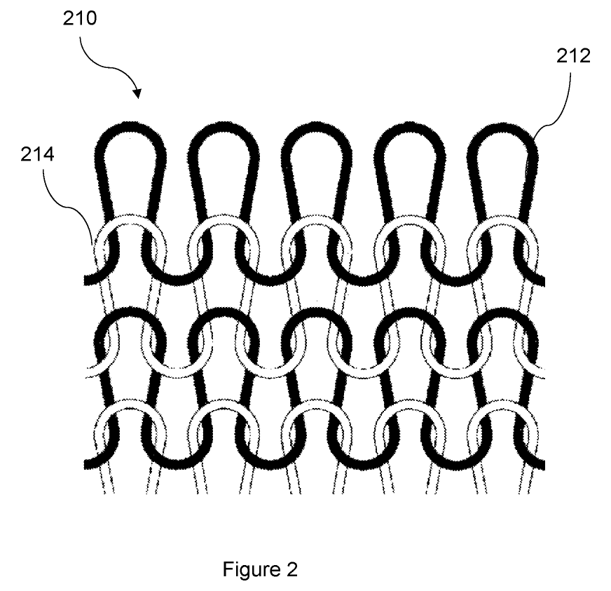

The functional fabric 100 may comprise a composite fabric layer 110, formed of knitted fibres or yarns. An example section of a knitted composite fabric layer 210 is illustrated in FIG. 2. Whilst the example illustration of FIG. 2 is of a double jersey knitted structure, it will be understood that any suitable knitting structure may be contemplated.

The composite fabric layer 210 may comprise first hydrophilic yarns 212, and second hydrophobic yarns 214, where the yarns may be knitted in a structure or pattern forming the composite fabric layer. Various methods of knitting include, but are not limited to, flat knitting, weft knitting, or warp knitting. For example, the hydrophilic yarns 212 and the hydrophobic yarns 214 may be knitted together as a composite or unitary layer, such as composite layer 110 as shown in FIG. 1A, that may have wicking and absorbent functions, as well as stain release properties as outlined herein. The hydrophilic yarns 212 and the hydrophobic yarns 214 may be knitted by warp knitting such as Raschel or Tricot, weft knitting (such as circular or flat), weaving, non-woven methods (blow spinning, staple non-woven, spun laid, air-laid, needle punched, thermal bonded, hydro-entangled, chemical bonded and so forth), electro-spinning, force-spinning, etc.

As set out herein, the liquid impermeable barrier 140 as shown in FIG. 1A may form at, or be formed of, a surface of the composite layer 210, 110, or may be attached to the composite layer 210, 110, such as by screen printing, glue lamination, or ultrasonic lamination, a sol-gel process, a vapour deposition process, a spray process, an electro-spray Ionisation, and/or a plasma vapour deposition. It will be understood that the liquid impermeable barrier as described herein may be formed, applied and/or attached via any other method to induce a substantially hydrophobic function.

The hydrophilic yarns 212 and/or hydrophobic yarns 214 may comprise fibres, or yarns made with fibres or filaments, where said fibres and/or yarns are selected from one or more of the group consisting of polyamide, polyester, polyolefin, polyurethane, polyacrylonitrile, natural cellulose, regenerated cellulose, regenerated cellulose derivatives (i.e. cellulose acetate and cellulose triacetates), natural protein and regenerated protein.

In an example embodiment, the hydrophilic yarns 212 may comprise naturally moisture-absorbing and/or moisture-wicking material, and/or may be treated to become moisture-absorbing and/or moisture-wicking. For example, the hydrophilic yarns 212 may be 100% polyester fabric, blends of polyester, polypropylene, polyethylene, nylon, regenerated materials (e.g. viscose blends), natural fibres (e.g. bamboo), and cotton. Additionally, the hydrophilic yarns 212 may also include one or more coatings, treatments, encapsulation, or entrapments, which would enhance its liquid and moisture management functionality, such as rate of wicking, wicking capacity, rate of spreading and distribution, one-way liquid/moisture transport, wetting time, moisture/liquid absorption rate, spreading speed of liquid, etc.

In an example embodiment, the hydrophobic fibres or yarns 214 may comprise one or more yarns provided with a treatment or coating to provide for a water repellent or partially water repellent property of the yarns. For example, the hydrophobic yarns 214 may be treated with a hydrophobic material or composition (for example, but not limited to: silicones, polyfluoroalkyl acrylates, polyacrylates, polyurethanes, or waxes) to create a hydrophobic yarn or fibre. Additionally, or alternatively, the hydrophobic yarns 214 may also include one or more coatings, treatments encapsulation or entrapments, which would enhance the liquid and moisture repellence of the hydrophobic yarns 214 (for example, but not limited flouro-polymers, non-fluoro polymers, ZnO (zinc oxide), silica). Additionally, or alternatively, the hydrophobic yarns 214 may comprise a surface modification to provide for liquid and moisture repellence, for example, plasma treatments or nano-coatings providing for a fibre or yarn surface that is substantially hydrophobic, liquid, or moisture repellent. It will be understood that other hydrophobic treatment or coating processes are present such that a fibre or yarn treated or coated with said processes comprises an increased hydrophobicity.

In an example embodiment, the ratio of hydrophilic yarns to hydrophobic yarns may be varied. For example, the composite fabric layer 210 may comprise a ratio of 50% hydrophilic yarns 212 and 50% hydrophobic yarns 214. However, in alternative embodiments the composite fabric layer 210 may comprise any ratio from 0-100% hydrophilic yarns and 0-100% hydrophobic yarns. Further, in example embodiments the composite fabric layer may comprise a gradient or varying ratio of hydrophilic yarns to hydrophobic yarns in a direction traverse along the fabric, across the fabric, through the fabric, or any combination of the above.

In an example embodiment, the functional fabric 100 and/or the composite fabric layer 110, 210, may be arranged such that the user facing side 120 comprises an arrangement, ratio or grouping of first hydrophobic yarns 214, and second hydrophilic yarns 212, and the outer facing side 130 comprises substantially hydrophobic yarns 214, or only hydrophobic yarns 214. Put another way, a ratio of hydrophobic yarns 214 to hydrophilic yarns 212 may be higher at the outer facing side 130 such that there are more hydrophobic yarns 214 toward the outer side 130. In this way, the hydrophobic yarns 214 forming or at the outer facing side 130 may form or comprise the substantially liquid impermeable barrier 140.

In an example embodiment, the functional fabric 100 as described herein may comprise a material, such as a single jersey, double jersey, or thin terry like, material with alternating hydrophobic yarns 214 and hydrophilic yarns 212. The functional fabric 100, or composite fabric layer 110 may comprise a linear array of hydrophobic yarns 214 and hydrophilic yarns 212, and/or alternating lines of hydrophobic yarns 214 disposed on top of or within the hydrophilic yarns 212. Advantageously, alternating hydrophobic yarns 214 and hydrophilic yarns 212 may enable stain release properties, whilst also enabling moisture management functions, such as moisture absorption and wicking.

Prior stain-resistant, stain release, or anti-stain fabrics may comprise hydrophobic materials or surfaces, which may be completely hydrophobic to avoid the forming of bonds and hence avoid staining. However, the completely hydrophobic nature of prior stain-resistant fabrics limits the ability for said material to absorb or wick liquids or fluids. Advantageously, the invention as disclosed herein may comprise a functional fabric, such as an absorbent fabric, formed with a ratio or composition of hydrophobic portions (such as hydrophobic yarns 214) and hydrophilic portions (such as hydrophilic yarns 212). This may result in improved stain release properties, and moisture management properties. For example, the arrangement of hydrophobic and hydrophilic portions may serve to break up a stain, allowing for a reduction in staining, and improvement in release of a stain. In another example, the hydrophobic yarns 214 may form numerous small hydrophobic barriers throughout a fabric that may prevent stains from deeply embedding into fibres of the fabric. For example, when a stain causing substance contacts a fabric, the hydrophobic yarns 214 may repel the stain-causing substance, which may limit their adherence. The limited adherence may help with lifting and carrying away stain particles during washing of the fabric. Additionally, the substantially liquid impermeable layer 140 may provide for a retention of any absorbed moisture or fluid within the functional fabric 100 and/or composite layer 110.

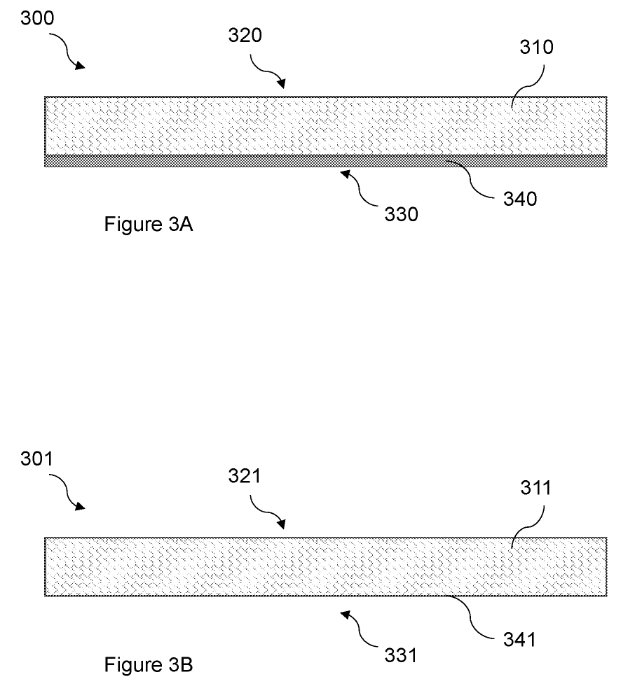

FIG. 3A shows a cross section of an example embodiment of a functional fabric 300 as described herein. The functional fabric 300 includes a composite layer 310, which may comprise an arrangement of hydrophilic yarns 212 and hydrophobic yarns 214 as described herein, such as in regard to FIG. 2. The functional fabric 300 may also comprise a liquid impermeable barrier layer 340, wherein the composite fabric layer 310 is attached to the liquid impermeable barrier layer 340. In an example embodiment, the liquid impermeable barrier layer 340 may be attached to the functional fabric 300 at an outer facing side 330, which is opposite to a user facing side 320 as described herein. Specifically, the composite fabric layer 310 is attached to the liquid impermeable barrier layer 340, at an outer facing side 330, such that liquid absorbed by the functional fabric 300 and/or composite layer 310 may be substantially prevented from leaking out of the functional fabric 300 and/or composite layer 310 by the liquid impermeable barrier layer 340.

In an example embodiment as shown in FIG. 3A, the liquid impermeable barrier layer 340 may comprise a print. The print may comprise a substantially thin layer, which may be secured to the functional fabric with adhesive. Alternatively, or in addition, the print may be a screen print, and may be a hydrophobic screen print. Alternatively, or in addition, the liquid impermeable barrier layer may comprise a chemical application, which may be a print and/or a coating. In an example embodiment, the coating or print may comprise the application of a very thin layer of material onto the surface of a substrate, such as the functional fabric as disclosed herein. In an example embodiment, an application method of the liquid impermeable barrier layer may comprise screen printing, such as flat-bed screen printing or rotary screen printing. The print or coating as described herein may comprise multiple layers of material and/or multiple layers or applications of a print, material and/or chemical.

In an example embodiment, the print or coating as described herein, which may form a liquid impermeable barrier, such as liquid impermeable barrier layer 340, may comprise a pattern, artwork or design applied to the functional fabric as disclosed herein, for example in the form of a print or screen print, whilst also providing for the hydrophobicity as disclosed herein.

As used herein, the term “adhesive” or “adhesive material” is a material that enables objects to be adhered to each other. For example, the adhesive material may include a material that becomes liquid impermeable after curing, such as a thermoplastic or thermoset glue. For example, the adhesive material may be in the form of liquid glue, such as hot-melt glue, drop-on-demand glue, line-on-demand glue, liquid resin, or the like, or combinations thereof. For example, the adhesive material may be in the form of an adhesive film or tape, such as a single-sided or double-sided adhesive film or tape. The term “print”, “screen print”, “printing”, or “coating” may refer to the application of a substance or material to a substrate or fabric. This may include the use of a stencil to selectively apply the substance or material to the fabric. In an example embodiment, the adhesion may refer to the adhesion of a substance or material by bonding directly with the substrate. In example embodiments this could comprise mechanical or chemical bonding. In some example embodiments, for example when additional adhesion is required, a primer or adhesive may be used to enhance the bonding between the printed material or substance and the fabric or substrate. In some example embodiments, a binding agent may be used to provide adhesion as described herein. For example, a fabric, such as the functional fabric as described herein, may be coated with a binder and a cross-linker, which may enable a chemical to bond to the fabric. In an example embodiment, the chemical may comprise a print or coating as described herein to provide hydrophobicity, such as to provide for the liquid impermeable barrier layer 340 as described herein.

In an example embodiment as shown in FIG. 3B, the functional fabric 301 includes a composite layer 311, which may comprise an arrangement of hydrophilic yarns 212 and hydrophobic yarns 214 as described herein, such as in regard to FIG. 2. The functional fabric 301 may also comprise a liquid impermeable barrier region 341, wherein the composite fabric layer 311 forms part of the liquid impermeable barrier region 341 at an outer facing side 331 of the fabric. Specifically, the composite fabric layer 311 may form the liquid impermeable barrier region 341 at an outer facing side 331 by comprising substantially hydrophobic yarns 214 disposed or positioned at the outer facing side 331. As outlined herein, this may be accomplished by a gradient or variation in the composition of the composite layer 311, for example, a composition graduating from a mixture of hydrophilic yarns 212 and hydrophobic yarns 214 at or towards a user facing side of the fabric 321, to substantially or only hydrophobic yarns 214 at an outer facing side 331 of the fabric. In this way, liquid absorbed by the functional fabric 301 and/or composite layer 311 may be substantially prevented from leaking out of the functional fabric 301 and/or composite layer 311 by the liquid impermeable barrier region 341.

In an example embodiment as shown in FIG. 3C, the functional fabric 302 includes a composite layer 312, which may comprise an arrangement of hydrophilic yarns 212 and hydrophobic yarns 214 as described herein, such as in regard to FIG. 2. In an alternative arrangement, the embodiment of FIG. 3C may comprise a composite layer 312 comprising only hydrophilic yarns 212. The functional fabric 302 may also comprise a liquid impermeable barrier layer 342, wherein the composite fabric layer 312 is attached to the liquid impermeable barrier layer 342, such as described above in relation to FIG. 3A.

Further, in the example embodiment of FIG. 3C, an additional substantially liquid impermeable barrier layer 352 may be attached or disposed at or towards the user facing side 322 of the functional fabric 302 and/or composite layer 312. In an example embodiment, the additional liquid impermeable barrier layer 352 may be attached to the functional fabric 302 at the user facing side 322, and may be attached as a complete layer, or at selected areas of the user facing surface 322 of the fabric. As outlined above, the additional substantially liquid impermeable barrier layer 352 may also be a print. The print may comprise a substantially thin layer or sections, which may be secured to the functional fabric with adhesive. Alternatively, or in addition, the print may be a screen print, and may be a hydrophobic screen print or sections of screen print.

In this way, the additional substantially liquid impermeable barrier layer 352 may provide for hydrophobicity as described herein, for example if combined with a composite layer 312 comprising only hydrophilic yarns 212, or a combination of hydrophilic and hydrophobic yarns. In this way, the abovementioned advantages of stain resistance and release, as well as moisture or fluid absorbency and wicking may be accomplished with the additional substantially liquid impermeable barrier layer 352 providing the hydrophobic region at or towards the user facing side 322 of the fabric.

In a similar way to FIG. 3A described above, the embodiment of FIG. 3C may also comprise a substantially liquid impermeable barrier layer 342, disposed at an outer facing side 332, such that liquid absorbed by the functional fabric 302 and/or composite layer 312 may be substantially prevented from leaking out of the functional fabric 302 and/or composite layer 312 by the liquid impermeable barrier layer 342.

The example embodiment as shown in FIG. 3D, the functional fabric 303 includes a composite layer 313, which may comprise an arrangement of hydrophilic yarns 212 and hydrophobic yarns 214 as described herein, such as in regard to FIG. 2. The functional fabric 303 may also comprise a liquid impermeable barrier region 343, wherein the composite fabric layer 313 forms part of the liquid impermeable barrier region 343 at an outer facing side 333 of the fabric. Specifically, the composite fabric layer 313 may form the liquid impermeable barrier region 343 at an outer facing side 333 by comprising substantially hydrophobic yarns 214 disposed or positioned at the outer facing side 333. As outlined herein, this may be accomplished by a gradient or variation in the composition of the composite layer 313, for example, a composition graduating from a mixture of hydrophilic yarns 212 and hydrophobic yarns 214 at or towards a user facing side of the fabric 323, to substantially or only hydrophobic yarns 214 at an outer facing side 333 of the fabric. In this way, liquid absorbed by the functional fabric 303 and/or composite layer 313 may be substantially prevented from leaking out of the functional fabric 303 and/or composite layer 313 by the liquid impermeable barrier region 343.

Further, in the example embodiment of FIG. 3D, an additional substantially liquid impermeable barrier layer 353 may be attached or disposed at or towards the user facing side 323 of the functional fabric 303 and/or composite layer 313. In an example embodiment, the additional liquid impermeable barrier layer 353 may be attached to the functional fabric 303 at the user facing side 323, and may be attached as a complete layer, or at selected areas of the user facing surface 323 of the fabric. As outlined above, the additional substantially liquid impermeable barrier layer 353 may also be a print. The print may comprise a substantially thin layer or sections, which may be secured to the functional fabric with adhesive. Alternatively, or in addition, the print may be a screen print, and may be a hydrophobic screen print or sections of screen print.

In this way, the additional substantially liquid impermeable barrier layer 353 of FIG. 3D may provide for hydrophobicity as described herein, for example if combined with a composite layer 313 comprising only hydrophilic yarns 212, or a combination of hydrophilic and hydrophobic yarns. In this way, the abovementioned advantages of stain resistance and release, as well as moisture or fluid absorbency and wicking may be accomplished with the additional substantially liquid impermeable barrier layer 353 providing the hydrophobic region at or towards a user facing side 323 of the fabric.

In a similar way to FIG. 3B described above, the embodiment of FIG. 3D may also comprise a substantially liquid impermeable barrier region 343 as set out above, disposed at an outer facing side 333 and formed as part of the composite layer 313, such that liquid absorbed by the functional fabric 303 and/or composite layer 313 may be substantially prevented from leaking out of the functional fabric 303 and/or composite layer 313 by the liquid impermeable barrier region 343.

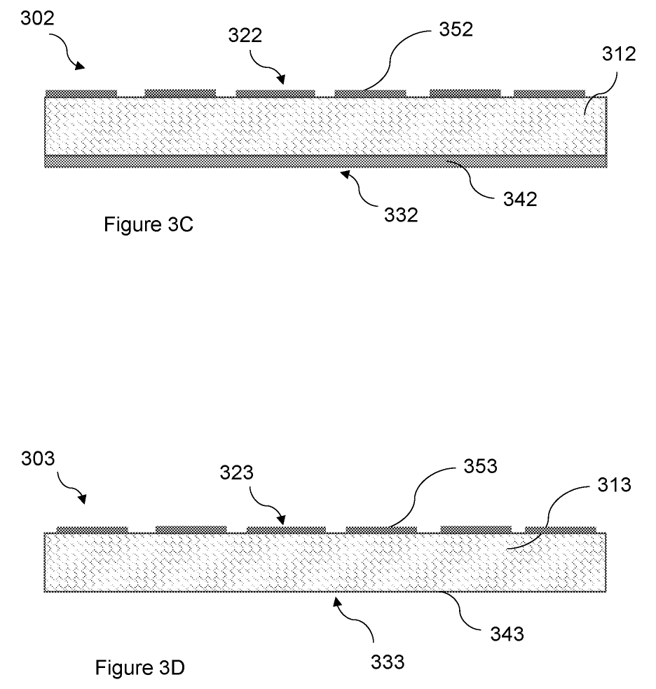

FIG. 4 illustrates an example embodiment of a functional fabric 400 as described herein, optionally comprising composite fabric 410. The example embodiment of FIG. 4 is shown as a top down view, facing a user facing side 420, such as user facing side 120, or 320-323 as described herein. The functional fabric 400 and/or the composite fabric layer 410 may comprise an array of hydrophilic portions 412 and hydrophobic portions 414 as described herein, which may be hydrophilic and hydrophobic yarns, such as described in FIGS. 3A and 3B, or portions of substantially liquid impermeable barrier region disposed on a user facing side 420, such as described in FIGS. 3C and 3D. The array of hydrophilic portions 412 and hydrophobic portions 414 may form channels for guiding liquid flow, where the user facing side 420 may comprise an array of channels formed of alternating hydrophobic portions 414 and hydrophilic portions 412 for guiding liquid flow. In an example embodiment, the channels may comprise hydrophobic portions 414 confined by one or more hydrophilic portions 412, to entrain and guide liquid flow.

In an example embodiment, the user facing side 420 may be formed of hydrophilic yarns 412, and may comprise alternating lines of hydrophobic yarns 414 disposed on top of or within the hydrophilic yarns. Alternatively, the user facing side 420 may comprise islands formed of hydrophobic yarns within areas of hydrophilic yarns.

In an example embodiment, the user facing side 420 may be formed of hydrophilic regions 412, and may comprise alternating lines of substantially liquid impermeable barrier regions 414 disposed on top of or within the hydrophilic regions 412. Alternatively, the user facing side 420 may comprise islands formed of substantially liquid impermeable barrier region within areas of hydrophilic region. As outlined above, the substantially liquid impermeable barrier layer or region may be a print. The print may comprise a substantially thin layer or sections, which may be secured to the functional fabric with adhesive. Alternatively, or in addition, the print may be a screen print, and may be a hydrophobic screen print or sections of screen print.

In examples as described herein, the presence of hydrophobic yarns 414 and/or substantially liquid impermeable barrier regions 414 at the user facing side 420 may provide for a dry feeling when in contact with a user’s skin. For example, the hydrophobic nature of the hydrophobic regions results in a lack of liquid absorption at these regions, meaning this region may stay dry, and may therefore provide a dry feeling against a user’s skin.

The functional fabric 400 and/or composite layer 410 may be capable of acquiring and distributing liquid or moisture. Specifically, when a garment comprising the functional fabric 400 and/or composite layer 410 is worn by the user, the fabric faces the user’s body / skin on a user facing side 420, and may serve to transport bodily fluids produced by the user’s body. In other words, the fabric may transport liquid from its upper interface, i.e. the user facing side 420, which may be in direct contact with the user’s skin, to its lower interface (the outer facing side as described herein) or an alternative region of the fabric.

Additionally, differential capillary forces on either surface of the functional fabric 400 can be created by the fabric structure of the composite layer 410, where the lower surface (outer facing side) has smaller pore sizes in comparison to the upper surface (user facing side 420). This pore combination may create funnel-like structures through the fabric in cross-section, where liquid is pulled from the user facing side with the larger pore sizes to the outer facing side with the smaller pore sizes, due to differential capillary pressure.

The rate of wicking through the functional fabric 400 may be controlled to be faster or slower. The rate may be set at a maximum rate of absorption of the functional fabric 400 to ensure that all, substantially all, or at least a significant percentage of, the liquid is absorbed by the functional fabric 400 and does not leak beyond the confines of the functional fabric 400. The rate of wicking may be controlled by the density, thickness, or composition of the functional fabric 400 of composite layer 410 and/or by the amount and type of hydrophobic and/or hydrophilic material applied to or forming part of the functional fabric 400 and/or composite layer 410. In an example embodiment, the rate of wicking may be set such that the user facing side 420 of the functional fabric 400 that may be in direct contact with the user feels "dry" or mostly dry to the user.

In some embodiments, the functional fabric 400 may include an antimicrobial agent or substance. For example, the antimicrobial substance may be one or more substances selected from the group consisting of a silver-containing substance, titanium dioxide, a quaternary silane, hydrogen peroxide, triclosan, and zinc pyrithione. Additionally, or alternatively, the functional fabric 400 may include an anti-odour agent or substance that combats odour. For example, the substance that combats odour may be one or more substances selected from the group consisting of nanoparticles with acid-neutralising pockets, high surface area mineral compositions, high surface area ceramic compositions, high surface area clay compositions, and plant-based deodorizers such as essential mint oils.

Further, additionally or alternatively, the functional fabric 400 may include a stain-resistant, stain-release, or stain-proof agent or substance, or may be formed to be stain-resistance, stain-release, or stain-proof via the composition of hydrophilic and hydrophobic material forming the functional fabric 400 as outlined herein. For example, the alternating nature of the functional fabric 400, with alternating hydrophilic regions 412 and hydrophobic regions 414, may serve to break up a stain or liquid that may cause a stain, such that a stain is less visible when absorbed, and/or easier to remove via washing. In an example embodiment, the functional fabric as disclosed herein may comprise a moisture management finish, which may comprise the composition of hydrophilic and hydrophobic material as described herein.

As set out above, and as shown in FIG. 4, the functional fabric 400 includes an array of hydrophobic regions or portions 414 and hydrophilic regions or portions 412 that form channels for guiding liquid flow. In an example embodiment, the hydrophobic regions 414 repel water towards the adjacent hydrophilic regions 412, which guide absorbed liquid to flow along those hydrophilic regions 412 and thereby improve liquid transport across the functional fabric 400. The hydrophobic regions 414 may be yarns treated to be liquid repellent, or substantially liquid impermeable barrier regions or layers applied to functional fabric 400, for example on a user facing side 420. The hydrophilic regions 412 may comprise yarns to collectively form channels that guide liquid flow. These channels may allow for greater movement and spread of liquid over a larger surface area for quicker evaporation. It will be understood that any movement or guiding of liquid flow as described herein is not limited to a specific direction and/or orientation. In an example embodiment, the channels as described herein, formed of alternating hydrophobic and hydrophilic regions, may comprise an artwork or design applied to the functional fabric as disclosed herein.

The liquid-repelling properties of the hydrophobic regions 414, along with the composition of the functional fabric to include a composite formed of hydrophobic portions 414 and hydrophilic portions 412, may also provide stain-free / stain-release properties as set out herein that may enhance aesthetics or introduce a visual effect to the functional fabric 400.

The hydrophilic region 412 may include any liquid absorbent / absorbing material known in the art (e.g. cotton, a cotton blend, foam, a synthetic material, absorbent polymeric foam, a nanotechnology-based or -produced material, or any other moisture-absorbent material). The material may have a weight of 50 to 900 g/m2, such as 180 to 300 g/m2.

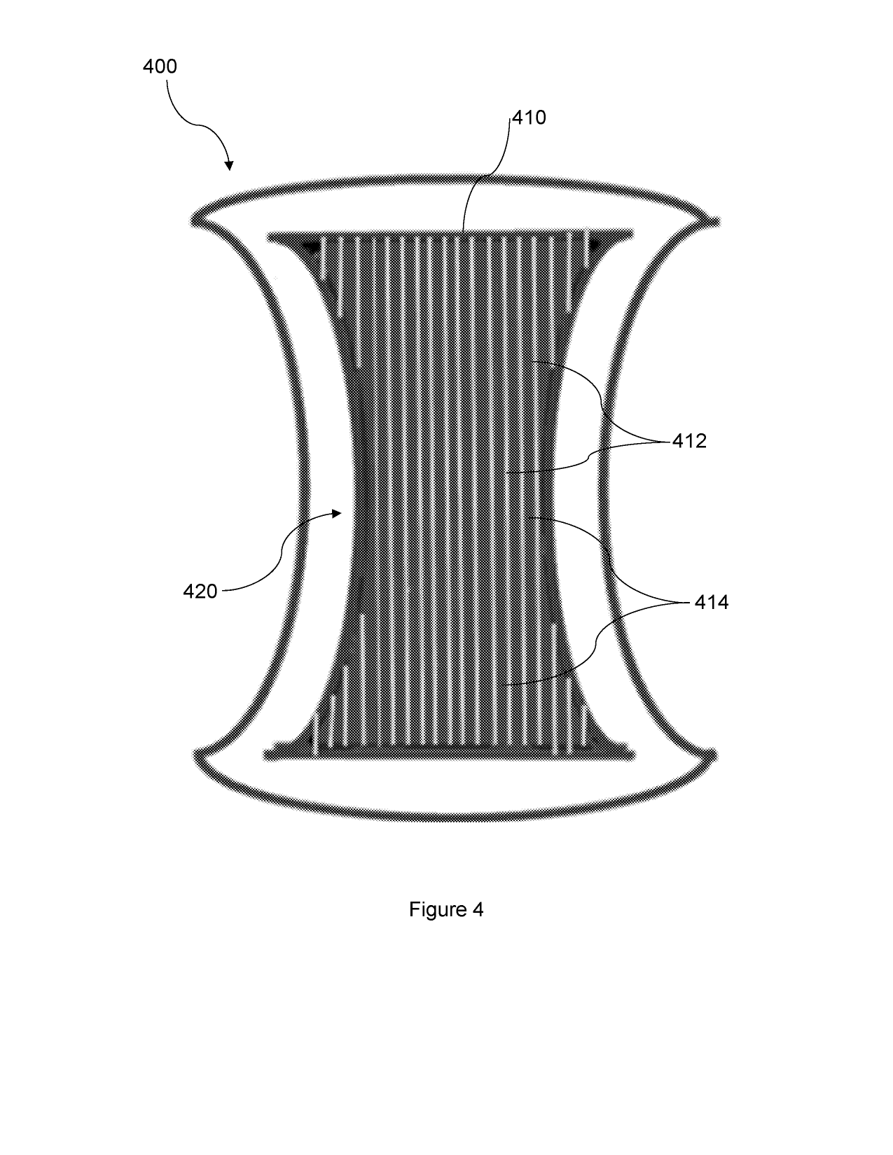

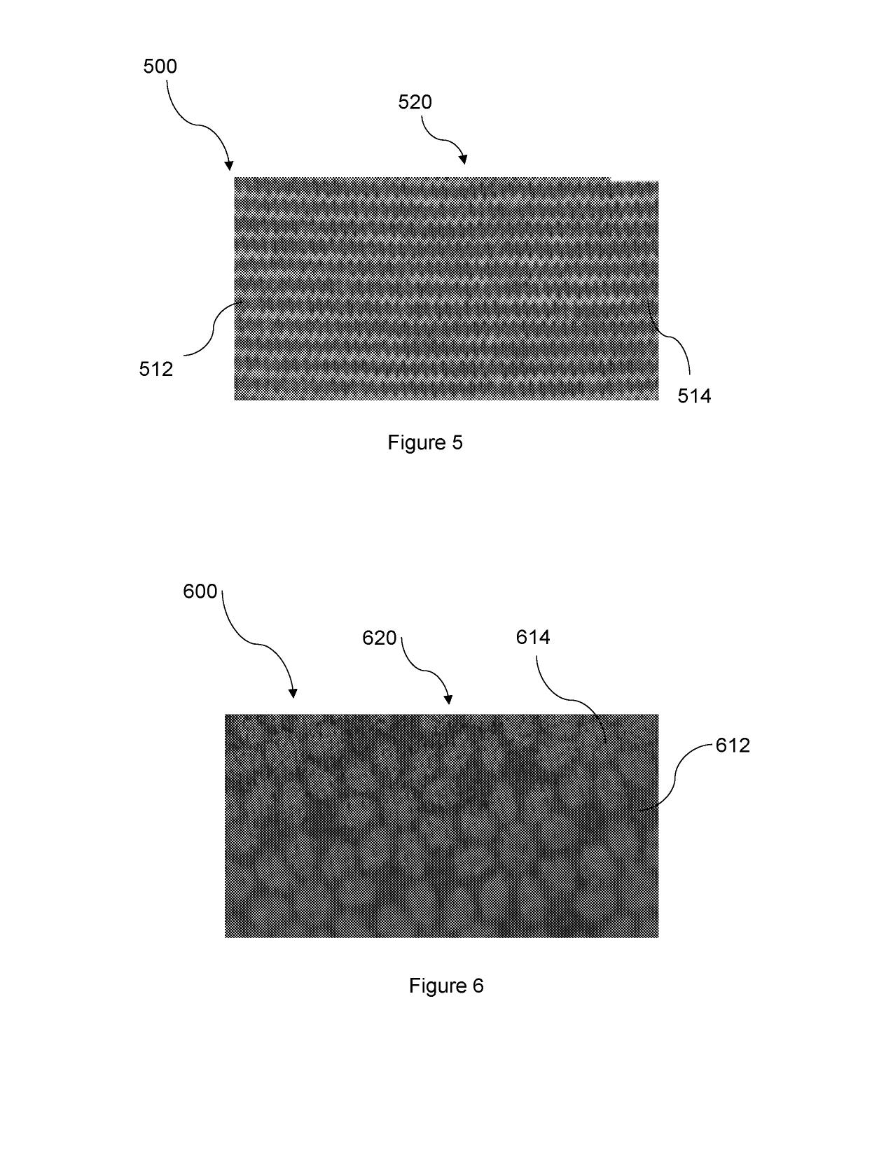

FIGS. 5 and 6 illustrate alternative embodiments of a functional fabric 500, 600 such as that described herein. Both FIGS. 5 and 6 show top down views of functional fabrics 500, 600, showing a user facing side 520, 620, respectively.

FIG. 5 shows an example embodiment of an alternately knitted woven functional fabric 500. As shown, the functional fabric 500 is formed of alternating zig-zag lines of hydrophobic regions 514, alternately woven with hydrophilic regions 512. In an example embodiment, the hydrophobic regions 514 may comprise hydrophobic yarns as described herein, and similarly the hydrophilic regions 512 may comprise hydrophilic yarns as described herein, forming a composite layer. Alternatively, the hydrophobic regions 514 may comprise substantially liquid impermeable barrier regions 514, which may be applied or printed to user facing side 520, for example onto a substantially hydrophilic fabric yarn base 512.

FIG. 6 shows an example embodiment of an alternately knitted woven functional fabric 600. As shown, the functional fabric 600 is formed of islands or regions of hydrophobic regions 614, alternately woven with hydrophilic regions 612. In an example embodiment, the hydrophobic regions 614 may comprise hydrophobic yarns as described herein, and similarly the hydrophilic regions 612 may comprise hydrophilic yarns as described herein, forming a composite layer. Alternatively, the hydrophobic regions 614 may comprise substantially liquid impermeable barrier regions 614, which may be applied or printed to user facing side 620, for example onto a substantially hydrophilic fabric yarn base 612.

Whilst zig-zag and island patterns are shown respectively in FIGS. 5 and 6 it will be understood that any pattern or composition of hydrophobic and hydrophilic regions may be provided as described herein. It will be understood that the functional fabric as disclosed herein is not limited to a specific design, pattern, and/or colour in the arrangement of hydrophilic and hydrophobic regions, and may take any form as required to accomplish the advantages as set out herein, including moisture management and/or stain resistance.

The example embodiments of both FIGS. 5 and 6 may comprise hydrophilic regions 512, 612 provided in a different colour, texture and/or pattern to hydrophobic regions 514, 614. The colour, texture and/or pattern of the hydrophilic regions 512, 612 may vary depending on a saturation of the respective region with absorbed liquid. In this way, a colour, texture and/or pattern may vary between the hydrophilic regions 512, 612 and hydrophobic regions 514, 614 depending on a liquid absorbency level of the functional fabric 500, 600, which may provide for an indicator of a dry or wet functional fabric 500, 600. For example, the hydrophilic region 512, 612 may become darker (alternatively lighter) when wet, which may reduce (alternatively increase) a contrast between the hydrophobic region 514, 614, depending on a colour chosen for each of the hydrophobic region and hydrophilic region. Advantageously, this may provide for an identification of saturation level of the functional fabric 500, 600, and may result in the functional fabric 500, 600 returning to its original appearance when dried.

In an example embodiment, a pattern formed on the functional or absorbent fabrics disclosed herein, which may be formed by a composition of hydrophobic and hydrophilic yarns, and/or a combination of hydrophilic yarns and substantially liquid impermeable barrier regions positioned on a user facing side, may be enhanced when the functional or absorbent fabric absorbs liquid, or alternatively may reduce or disappear, as the hydrophilic regions darken or lighten due to moisture absorption.

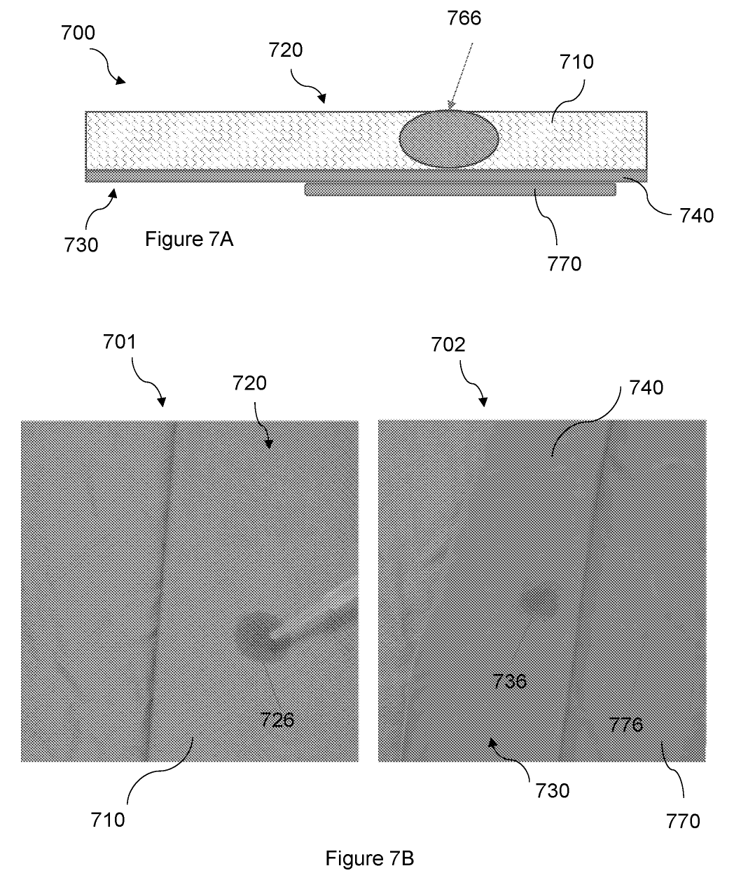

FIGS. 7A and 7B illustrate an example embodiment of a functional fabric arrangement as described herein, along with experimental results of liquid absorption of the example arrangement.

FIG. 7A shows a cross section of an example embodiment of a functional fabric 700 as described herein. The functional fabric 700 includes a composite layer 710, which may comprise an arrangement of hydrophilic yarns 212 and hydrophobic yarns 214 as described herein, such as in regard to FIG. 2. The functional fabric 700 may also comprise a liquid impermeable barrier layer 740, wherein the composite fabric layer 710 is attached to the liquid impermeable barrier layer 740. In an example embodiment, the liquid impermeable barrier layer 740 may be attached to the functional fabric 700 at an outer facing side 730, which is opposite to a user facing side 720 as described herein. Specifically, the composite fabric layer 710 may be attached to the liquid impermeable barrier layer 740, at an outer facing side 730, such that liquid absorbed by the functional fabric 700 and/or composite layer 710 may be substantially prevented from leaking out of the functional fabric 700 and/or composite layer 710 by the liquid impermeable barrier layer 740. This is shown experimentally with the results of FIG. 7B.

FIG. 7B shows left 701 and right 702 images, showing respectively the user facing side 720, and outer facing side 730 of a functional fabric 700.

FIG. 7A illustrates a liquid 766 applied to functional fabric 700, which has been absorbed into functional fabric 700 via the composite layer 710 formed of hydrophilic and hydrophobic portions as described herein. Further, the liquid 766 has stopped and been held within the functional fabric 700 by the combination of the composite layer 710, and the liquid impermeable barrier layer 740, leading to reduced or no leakage of fluid through the functional fabric 700.

This is shown in FIG. 7B, where user facing side 720, on left image 701, shows the liquid 726 applied and wicked into functional fabric 700. Similarly, the right image 702 shows the outer facing side 730, including liquid impermeable barrier layer 740, where liquid is visible at 736, but has not passed through the liquid impermeable barrier layer 740 onto the outer facing side 730. This is shown by blotting paper 770, which was placed as shown in FIG. 7A, in contact with liquid impermeable barrier layer 740 when liquid was applied to functional fabric 700, and then removed, as shown in FIG. 7B, right image 702, where blotting paper 770 shows a dry region 776 that was in contact with liquid impermeable barrier layer 740 at 736, and has therefore not been in contact with fluid (indicating no leakage beyond the liquid impermeable barrier layer 740).

Many embodiments of the functional or absorbent fabric 100, 300-303, 400, 500, 600 and 700, as well as the garment 190 are described in the present disclosure. It will be appreciated that various aspects of one or more embodiments of the functional or absorbent fabric and/or garment may apply equally to another one or more other embodiments, and such aspects are not further elaborated upon for purpose of brevity.

In the foregoing detailed description, embodiments of the present disclosure in relation to a functional fabric and/or an absorbent fabric or pad for use in a garment and a garment comprising the functional fabric and/or an absorbent fabric or pad are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

What is claimed is:

1. A functional fabric for use in a garment, the functional fabric comprising:
first hydrophobic yarns and second hydrophilic yarns arranged to form a composite fabric layer, the composite fabric layer comprising a user facing side and an outer facing side,
wherein the outer facing side comprises a liquid-impermeable barrier, and
wherein the functional fabric further comprises a hydrophobic coating disposed on at least a portion of the outer facing side of the composite fabric layer to form the liquid-impermeable barrier.

2. The functional fabric of claim 1, wherein the functional fabric forms at least part of a gusset of an undergarment.

3. The functional fabric of claim 1, wherein the functional fabric is leak proof or liquid impermeable.

4. The functional fabric of claim 1, wherein the composite fabric layer is arranged such that the user facing side comprises an arrangement of first hydrophobic yarns and second hydrophilic yarns, and the outer facing side comprises only hydrophobic yarns.

5. The functional fabric of claim 1, wherein the composite fabric layer comprises a ratio of hydrophilic yarns to hydrophobic yarns, wherein the ratio of hydrophilic yarns to hydrophobic yarns forming the composite fabric layer is 50/50, or wherein the quantity of hydrophilic yarn is between 20% and 80% of the composite fabric.

6. The functional fabric of claim 5, wherein the outer facing side of the composite fabric layer comprises a higher ratio of hydrophobic yarns to hydrophilic yarns than the user facing side.

7. The functional fabric of claim 5, wherein the ratio of hydrophilic yarns to hydrophobic yarns comprises a varying gradient extending from the first user facing side to the second outer facing side.

8. The functional fabric of claim 1, further comprising a hydrophobic print provided on at least a portion of the user facing side.

9. The functional fabric of claim 1, wherein one or more of the following applies:
(a) the composite fabric layer comprises a knit or weave of hydrophobic yarns and hydrophilic yarns;
(b) the composite fabric layer comprises a pattern of alternating hydrophobic and hydrophilic yarns; and
(c) the composite fabric layer comprises a linear array of hydrophobic yarns and hydrophilic yarns.

10. The functional fabric of claim 1, wherein the composite fabric layer comprises alternating lines of hydrophobic yarns disposed on top of or within the hydrophilic yarns, forming an array of hydrophobic yarns and hydrophilic yarns that form channels for guiding liquid flow.

11. The functional fabric of claim 1, wherein one or more of the following applies:
(a) the user facing side comprises an array of channels formed of alternating hydrophobic yarns and hydrophilic yarns for guiding liquid flow, the channels comprising a region of hydrophobic yarns confined by one or more regions of hydrophilic yarns;
(b) the user facing side comprises alternating lines of hydrophobic yarns disposed on top of or within the hydrophilic yarns;
(c) the composite fabric layer comprises islands formed of hydrophobic yarns within areas of hydrophilic yarns; and
(d) the composite fabric layer provides both a wicking and an absorbent function.

12. The functional fabric of claim 1, wherein the functional fabric is configured such that a colour of the hydrophilic yarns varies compared to a colour of the hydrophobic yarns when the functional fabric absorbs a liquid.

13. The functional fabric of claim 1, wherein one or more of the following applies:
(a) the hydrophilic yarns comprise a cotton yarn,
(b) the hydrophobic yarns comprise pre-treated cotton yarns, the cotton yarns pre-treated before a forming step of the composite material,
(c) the hydrophobic yarns comprise pre-treated cotton yarns, the cotton yarns pre-treated with a chemical treatment comprising a hydrophobic coating.

14. The functional fabric of claim 1, wherein the hydrophilic yarns provide for a wicking and/or absorbing region of the composite fabric layer.

15. The functional fabric of claim 1, wherein the hydrophobic yarns provide for a stain releasing function of the composite layer.

16. The functional fabric of claim 1, wherein the functional fabric is arranged to provide for a stain release or stain-resistant or anti-stain fabric.

17. The functional fabric of claim 1, wherein the user facing side provides for a stain release function.

18. The functional fabric of claim 1, wherein the functional fabric comprises an absorbent fabric or absorbent function.

19. A functional fabric for use in a garment, the functional fabric comprising:
one or more hydrophobic regions, and one or more hydrophilic regions, the hydrophobic region(s) and hydrophilic region(s) arranged to form a composite fabric layer, the composite fabric layer comprising a user facing side and an outer facing side, and
a liquid impermeable barrier layer or hydrophobic coating disposed on at least a portion of the outer facing side of the composite fabric layer,
wherein the liquid impermeable barrier layer or hydrophobic coating forms a liquid-impermeable barrier, to prevent or reduce seeping of a fluid through the outer facing side of the composite fabric layer.

20. The functional fabric of claim 19, wherein, when the functional fabric is incorporated with, or to, a garment, the liquid impermeable barrier layer prevents or reduces seeping of a fluid into the garment.
